(19) United States
(12) Patent Application Publication
(10) Pub. No.: US 2025/0347031 A1
(43) Pub. Date: Nov. 13, 2025

(54) MACHINE LEARNING PIPELINE FOR EFFICIENT EXPLORATION OF COMBINATORIAL SPACE

(71) Applicants: International Business Machines Corporation, Armonk, NY (US); The Regents of the University of California, Oakland, CA (US)

(72) Inventors: Sara Capponi, San Francisco, CA (US); Jie Shi, San Jose, CA (US); Shangying Wang, San Jose, CA (US); Jordan James Baker, Oakland, CA (US); John Eugene Dueber, San Francisco, CA (US)

(21) Appl. No.: 18/657,280

(22) Filed: May 7, 2024

(51) Int. Cl. C40B 30/00; C40B 40/08; C40B 40/10; G16B 35/20; G16B 40/00; G16C 20/64; G16C 20/70

(52) U.S. Cl. CPC C40B 30/00; C40B 40/08; C40B 40/10; G16B 35/20; G16B 40/00; G16C 20/64; G16C 20/70

(57) ABSTRACT

A computer-implemented method and related system explore a combinatorial space. A combinatorial library and a desired output are identified. From the combinatorial library, an initial dataset is identified to be tested experimentally to create the combinatorial space. The following functions are iteratively performed: experimentally screening a set of diverse machine learning models (MLMs) using the initial data set or an augmented data set to produce experimental screening results; training the MLMs using the experimental screening results; selecting, from the MLMs, at least one MLM having a highest accuracy and performance; screening the combinatorial library; calculating a normalized similarity factor measured from top-ranked combinations; identifying, using the normalized similarity factor, an amount of the model-driven augmented data to be added to the top-ranked combinations; obtaining augmented data; and selecting the augmented data from the top-ranked combinations and the augmented combinatorial data. The iteration exits upon meeting an exit criterion.

### GOVERNMENT LICENSE RIGHTS

This invention was made with government support under Prime Contract #DBI-1548297 awarded by the National Science Foundation. The government has certain rights in the invention.

### STATEMENT REGARDING PRIOR DISCLOSURES BY THE INVENTOR OR A JOINT INVENTOR

The following disclosure is submitted under 35 U.S.C. § 102(b)(1)(A):

### DISCLOSURE(S)

Jordan J. Baker, Jie Shi, Shangying Wang, Elena M. Mujica, Sara Capponi, and John E. Dueber, Improved Peroxisome Capacity Aided by Machine Learning Enables Improved Compartmentalization of a Multi-Enzyme Pathway, submitted for peer review to Nature Chemical Biology on Jan. 23, 2024.

### BACKGROUND

A system and method are provided for using a machine learning pipeline for efficient exploration of a combinatorial space.

### SUMMARY

Disclosed herein is a computer-implemented method for exploring a combinatorial space using one or more processors. The method comprises identifying a combinatorial library and a desired output. From the combinatorial library, an initial dataset is identified to be tested experimentally to create the combinatorial space from elements within the combinatorial library. The following functions are iteratively performed: 1) experimentally screening a set of diverse machine learning (ML) models (MLMs) using the initial data set or an augmented data set to produce experimental screening results that comprise experimental outputs; 2) training the MLMs using the experimental screening results; 3) selecting, from the MLMs, a selected MLM set (SMS) comprising at least one MLM having a highest accuracy and performance; 4) obtaining a set of top-ranked combinations by screening the combinatorial library using the SMS; 5) calculating a normalized similarity factor measured from the top-ranked combinations; 6) identifying, using the normalized similarity factor, an amount of the model-driven augmented data to be added to the top-ranked combinations; 7) obtaining a set of model-driven augmented data; and 8) selecting the augmented data set from the top-ranked combinations and the augmented combinatorial data. Upon meeting an exit criterion of the iterations, the method comprises determining an optimal combination identification that produces the desired output.

Furthermore, embodiments may take the form of a related system comprising hardware and software described herein for performing various operations of the method described above, and a computer program product, accessible from a computer-usable or computer-readable medium providing program code for use, by, or in connection, with a computer or any instruction execution system. For the purpose of this description, a computer-usable or computer-readable medium may be any apparatus that may contain a mechanism for storing, communicating, propagating or transporting the program for use, by, or in connection, with the instruction execution system, apparatus, or device.

### BRIEF DESCRIPTION OF THE DRAWINGS

Various embodiments are described herein with reference to different subject-matter. In particular, some embodiments may be described with reference to methods, whereas other embodiments may be described with reference to apparatuses and systems. However, a person skilled in the art will gather from the above and the following description that, unless otherwise notified, in addition to any combination of features belonging to one type of subject-matter, also any combination between features relating to different subject-matter, in particular, between features of the methods, and features of the apparatuses and systems, are considered as to be disclosed within this document.

The aspects defined above, and further aspects disclosed herein, are apparent from the examples of one or more embodiments to be described hereinafter and are explained with reference to the examples of the one or more embodiments, but to which the invention is not limited. Various embodiments are described, by way of example only, and with reference to the following drawings:

FIG. 1 is a block diagram of a general computing device and environment.

FIG. 2 is a block flow diagram illustrating an example process for an efficient exploration of combinatorial space, according to some embodiments.

FIG. 3 is a block diagram that illustrates a combinatorial library that defines a combinatorial space based on an identified initial data set.

### DETAILED DESCRIPTION

The following general acronyms may be used below:

TABLE 1
General Acronyms

| | |
|---|---|
| CD-ROM | compact disc ROM |
| CPP | computer program product |
| DVD | digital versatile disk |
| EPROM | erasable programmable read-only memory |
| EUD | end-user device |
| IoT | Internet of Things |
| LAN | local-area network |
| NFC | near field communication |
| RAM | random access memory |
| ROM | read-only memory |
| SAN | storage area network |
| SD | secure digital |
| SDN | software-defined networking |
| SRAM | static random-access memory |
| UI | user interface |
| USB | universal serial bus |
| VCE | virtual computing environment |
| WAN | wide-area network |

### Data Processing System in General

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

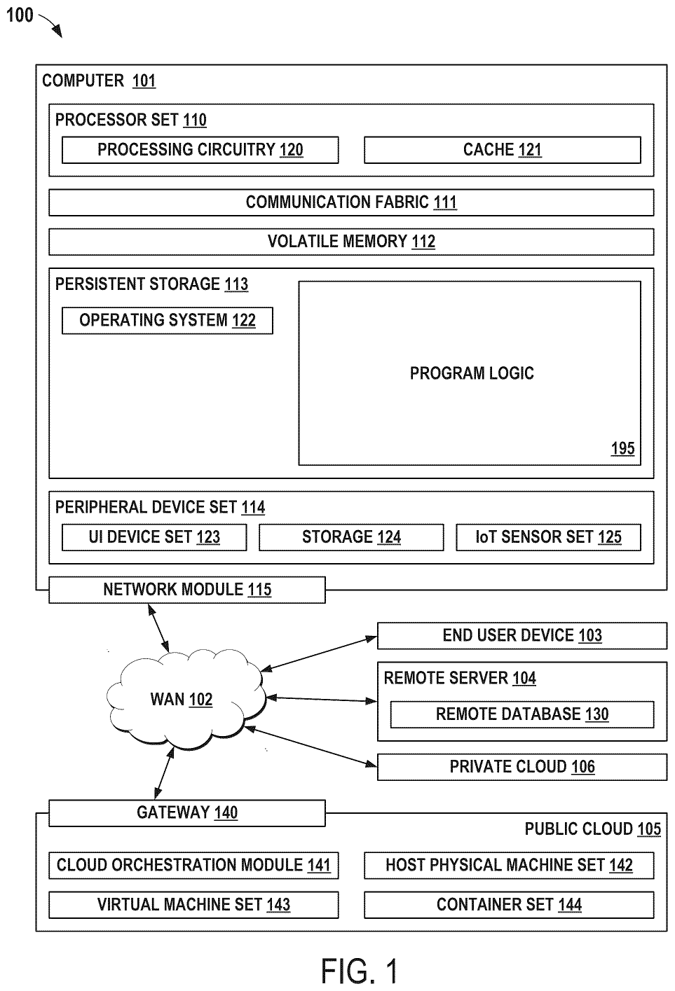

FIG. 1 is a block diagram of a general computing device and environment. Computing environment 100 contains an example of an environment for the execution of at least some of the computer code involved in performing the inventive methods disclosed herein, including program logic 195 that may be implemented in various combinations of hardware and/or software described below. In addition to the program logic 195, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and program logic 195, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in the program logic 195 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in the program logic 195 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economics of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

The descriptions of the various embodiments of the present invention are presented for purposes of illustration, but are not intended to be exhaustive or limited to the embodiments disclosed. Many modifications and variations will be apparent to those of ordinary skill in the art without departing from the scope and spirit of the described embodiments. The terminology used herein has been chosen to best explain the principles of the embodiments, the practical application or technical improvement over technologies found in the marketplace, or to enable others of ordinary skill in the art to understand the embodiments disclosed herein.

Certain reference numbers or characters may be represented as being pluralities (e.g., 100.1, 100.2, etc.). In such instances, reference to a single reference number (e.g., 100) may represent the plurality of entities, or may represent an example of the set, depending on the context. This similarly applies to reference numbers or characters that use subscripts.

### Machine Learning Pipeline for Efficient Exploration of Combinatorial Space

The following application-specific acronyms may be used below:

TABLE 2
Application-Specific Acronym

| | |
|---|---|
| ML | machine learning |
| MLM | machine learning model |

Engineering, biological, and chemical processes very often require exploring a vast combinatorial space to find an optimal combination of elements that provides a respective engineering, biological, or chemical solution with desired outcomes/functions. For example, in the field of biology, engineering proteins requires searching in the amino acid combinatorial space for the optimal sequence that will carry out specific functions. Designing cell phenotypes (i.e., observable characteristics of individuals based on a genotype interacting with the environment) requires searching for a combination of genes/proteins that will give rise to the desired phenotype.

Empirical exploration of the full, vast combinatorial space for elements, such as amino acids, requires testing experimentally all possible combinations of elements, posing a significant burden that is often times impossible or prohibitively expensive in terms of resources to solve. By using rational design approaches, it is possible to explore, experimentally, subregions of this vast combinatorial space. However, these rational design approaches are limited by a prior knowledge of the mechanism underlying the related elements, e.g., the gene/protein. Moreover, the optimal solution/outcome with desired functions might be located outside of such explored subregions, making such an optimal solution difficult to find.

Traditional approaches using methods based on active learning or a Bayesian ensemble are computationally expensive and use predictions with high uncertainty to enlarge the input dataset and avoid entrapment in local optima. However, the optimal solution with desired functions can be located outside the subregions defined by combinations of elements with high uncertainty, and thus these methods will not guarantee the efficient exploration of all uncharted regions of the combinatorial space.

In a very simplified example, a combinatorial space comprises two separate element sets, each containing five elements. The element sets are {A1 . . . . A5}, and {B1 . . . . B5}, and the combination rules are one element from each set. This means the combinatorial space may be represented by {A1B1 . . . . A1B5, A2B1 . . . . A2B5, . . . . A5B1 . . . . A5B5} for a total of twenty five (52) combinations. It can be seen that as the number of sets and elements within the sets increase, the combinatorial space can grow very quickly. For the same rules above, a combinatorial space having ten sets with ten values each will have 1010 combinations.

Using this simplified example, rational design approaches limited by a prior knowledge of the mechanisms underlying the related elements may suggest only combinations from the first two elements in each of the sets (A1B1 . . . . A2B2), i.e., four combinatorial elements. Furthermore, a local optimum may be present within these four combinatorial elements (e.g., A1B2). However, the optimal solution may be outside of these four combinatorial elements (e.g., A3B5).

Therefore, there is a need for developing a novel approach that can facilitate the exploration of the combinatorial space efficiently by leveraging the power of machine learning (ML) models (MLMs) and at the same time be less resources-intensive than the traditional approaches.

Previous approaches may leverage the predictive capabilities of ML models to identify optimal combinatorial libraries, however, they do so with a focus capability only on local subregions utilizing limited data to extrapolate for a larger input space. The method used according to various embodiments disclosed herein targets the entire combinatorial space by proactively augmenting input data at poorly explored subregions and maximizes the complementary strengths between experiments and ML models through an automated workflow. . . . At each iteration of the workflow, the ML models offer valuable guidance on which input combinations warrant investigation, while the experimental data provide essential ground truth for further enhancing prediction accuracy of ML models.

Other previous approaches may introduce an ML-based workflow to identify optimal combinatorial libraries. However, these approaches only apply the same neural network multiple times. The method used according to various embodiments disclosed herein systematically tests different MLMs and choose the top performers. The choice of the set of MLMs ensure that any type of data set can be analyzed and the analysis is not biased by the inherent architecture of the specific models.

This approach is advantageous over a system in which the workflow uses only one single MLM to analyze experimental data from the combinatorial space 305, which might not be the most suitable model for data different from that which a user chooses for implementing the workflow. In addition, these embodiments may make use of synthetic data to ensure an exploration of subregions of the combinatorial space related to bad performance combinations.

Given large input spaces, the methods of previous approaches tend to be trapped in suboptimal regions, which can affect the search. Even where existing solutions improve model performance and accuracy, they still do not utilize samples that are selected from uncharted regions of a combinatorial space, making the search for optimal result insufficient and less accurate. Various embodiments of the iterative pipeline disclosed herein aggressively look for new samples with optimal outcomes and for under-represented regions in a straightforward manner. The embodiments generate additional combinations of unrepresented or unseen elements ensuring exploratory sampling of the combinatorial space, which improves the model performance and escapes getting stuck in local optima. The method used according to various embodiments disclosed herein introduces an additional MLM selection algorithm to: a) ensure the balance of input data space; and b) help escape sub-optimal entrapment. This helps to avoid such entrapment and makes the final optimal selection less dependent on the initial library pick. Furthermore, the use of the various embodiments of the iterative pipeline disclosed herein follows an iterative procedure where the selected ensemble predictions are then tested experimentally, and the new experimental data are used once again for starting another cycle. Put differently, while ML models technically can screen the “all regions”, previous approaches rely on information/data at a local region and try to screen the entire region by extrapolation from a local region to the entire region, including the outer space. The MLM selection algorithm described herein proactively identifies those under-represented regions outside of the local data area, thus making the searching on the entire region more accurate and more efficient.

The novelty of this approach includes: i) utilizing ensemble predictions for a combination of elements from the combinatorial space via a set of diverse ML models; ii) constructing model-driven augmented data with combinations located in previously poorly explored regions of the combinatorial space; and iii) defining a similarity factor that guides an identification of new samples to be tested experimentally in a next iteration. Note that some elements of the combination may come from explored regions, but the explored regions are a small percentage of the total landscape with most data points falling into the poorly explored regions. Each single data point is a combination of elements, but the input data, the combinatorial point, is a high-dimensional realm. By way of example, the initial data may contain both element A and element B. However, the model may not see the two elements interact with each other. In that case, the algorithm disclosed herein may make suggestions of augmented data in which element A and B appear/interact together in the same data sample.

The ensemble predictions comprise a set (ensemble) of predictions that are made using the set of diverse ML models. This may be done by choosing a set of diverse ML models, i.e., ML models having distinctive architectures that make them inherently diverse and different from one to another. Then each single ML model in this set is trained and evaluated. The models having the top accuracy are then chosen for the following iterative searching. Thus, instead of having a single prediction, a set/ensemble of predictions are produced. This set of predictions can provide an indication of a range of outcomes in order to make probabilistic predictions for other sets of elements from the combinatorial space. The ML architecture explores a large combinatorial space utilizing a small initial data set and discovers combinations with optimally desired functionalities (e.g., phenotypes) that may then be tested experimentally.

By more efficiently sampling, predicting, and testing the combinatorial space, the process workflow may accelerate discoveries in any field where the desired function depends on a combination of element from a combinatorial library. The process described herein may be applied to a wide range of applications. The following is a non-exhaustive list of areas for use, which includes: i) the design of any biological property, including metabolic pathways and/or protein engineering, organelle engineering, protein interactions, etc.; ii) the discovery of new materials given by the combinations of different chemical elements belonging to a defined combinatorial library; and iii) screening for ligand binding, optimization of microbial strains, and building genetic circuits. This may specifically include, by way of example only, synthetic biology, designing new epitopes, proteins, interactions between chemicals and/or proteins, and novel chemical compounds among others.

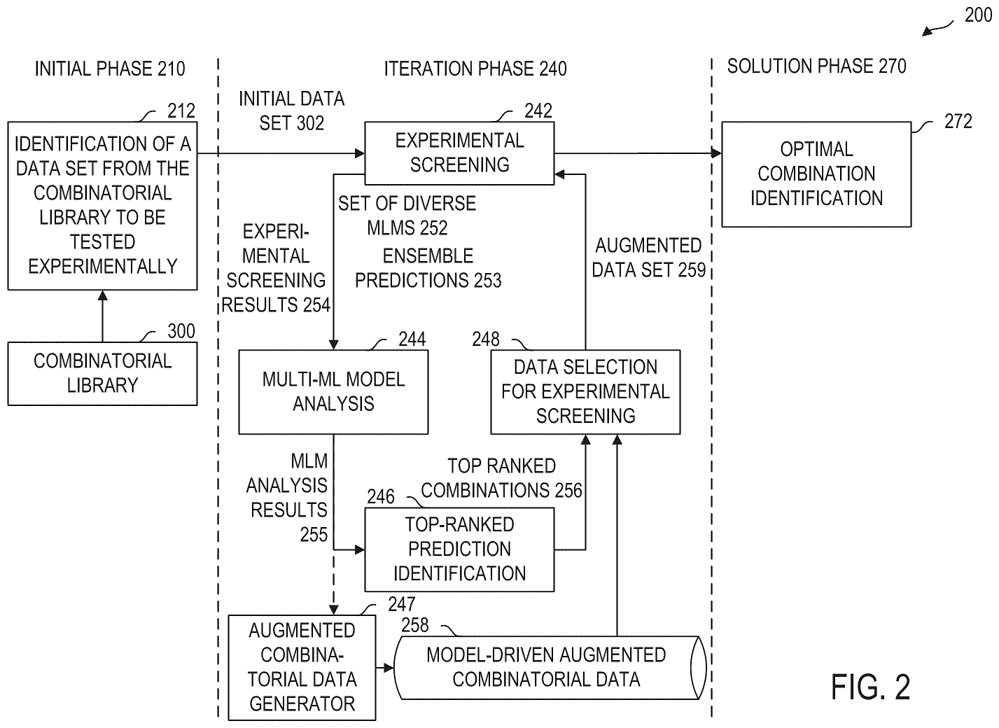

FIG. 2 is a block flow diagram illustrating an example process 200 for an efficient exploration of combinatorial space, according to some embodiments. The ML-based process 200 is pipeline that enables, more efficiently, the exploration of a vast combinatorial space by leveraging the power of experimental screening and ML-predictive MLMs applied in an iterative manner. The process 200 aims at examining comprehensively the combinatorial space and at identifying an optimal combination of elements to provide desired functions as an outcome.

The process 200 utilizes experimental testing and ML that is based on in silico screening (i.e., using computer modeling/simulation) to direct and iteratively improve the search for the optimal combination of elements with the desired trait/functions as an outcome. The input data are combinations of elements, with the combinations resulting in the combinatorial space being vast, yet finite. The iterative pipeline screens the entire combinatorial space and provides different recommendations without resourcing to computationally expensive algorithms.

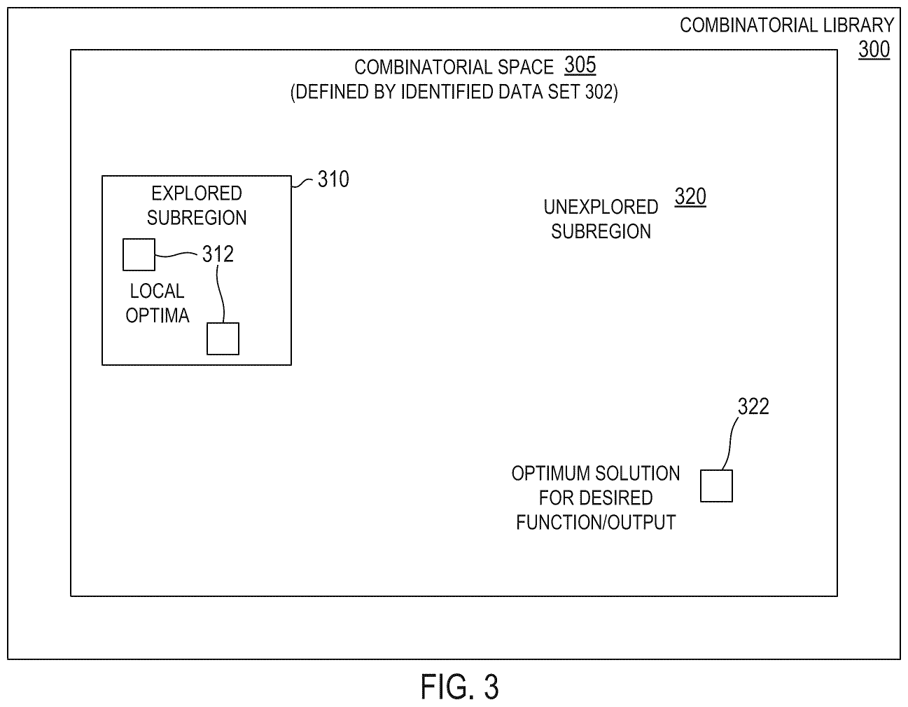

FIG. 3 is a block diagram that illustrates a combinatorial library 300 from which a combinatorial space 305 may be identified based on a specified initial data set 302. In prior solutions, an explored (or explorable) subregion 310 is limited to a fairly small portion of the combinatorial space 305. Although local optima 312 may be found within this explored/explorable subregion 310, the local optima 312 may not necessarily include the optimum solution 322, which may be located in the significantly larger unexplored region 320.

Referring back to the process 200, in an initial phase 210 (before the iterative operations), in an identification (first) operation 212, the data set 302 comprises elements identified from the combinatorial library 300 to be tested experimentally, and a desired function/output are defined. The combinatorial libraries may encompass any type of data (e.g., genes, proteins, chemical compounds, etc.), and the optimal combination of elements from the library 300 may be related to a specific desired function/output. Each combination of the combinatorial library elements is related to a magnitude that can be measured experimentally. The experimental output can be a value or a Boolean (e.g., 0 or 1, Tor F). The larger and more scattered the initial data set 302 is, the better it is.

In the Use Case, described in more detail below, the combinatorial library is a library of genes, and the identified data set 302 to be tested experimentally comprises peroxisome-related genes. The desired function is an increased cargo capacity for a heterologous protein.

Once the data set is identified (e.g., the peroxisome-related genes) and the initial combinations are defined, the iteration phase 240 begins with an experimental screening (fifth) operation 242. The operations of the iteration phase 240 may be significantly automated and does not require a prior knowledge of the specific problem being addressed. The automation aspect is achieved in that additional knowledge from outside the iteration phase is required—the output from the experiments is provided as the input in the MLMs' training, and the output of the MLMs goes into the input of the experiments. The computational process 200 may continuously improve via each iteration of the iteration phase 240 and may pause flexibly depending on a time/resource limit and a scope of the study.

In the experimental screening operation 242, a set suggested combinations of elements are provided by the diverse MLMs 252, which are selected to provide the ensemble predictions 253, i.e., taking the average of the predictions from the various MLMs to reduce variance. The set of diverse MLMs 252 may include, e.g., Scikit-learns® library models and customized deep learning models. The experimental screening accepts, as input, those top-predictions 256 and augmented data 259 and evaluate the actual performance (i.e., actual functions/phenotypes) to produce experimental screening results 254. Theses experimental screening results 252 constitute determined values that represent the real performance (ground truth) of the initial or current iteration combinations that ultimately serve as the input for the next iteration of MLMs 252 training. In the iteration phase 240, the set of diverse MLMs 252 may be trained using the initial data set 302 or an augmented data set 259, described in more detail below.

Given the inherent challenge of determining a priori which specific MLM is more suitable to learn from a specific experimental data set, the set different types of (i.e., diverse) MLMs 252 are used at the same time in parallel. At each iteration through the iteration phase 240, a set of different MLMs (e.g., scikit-learn library ML models combined with customized deep learning models) leverages the different architectures and learning approaches characteristic of the structure of each individual model and provides complementary strength and robustness in predictions.

In a multi-MLM analysis (second) operation 244, an MLM analysis is performed to produce MLM analysis results 255 generated by the multiple MLMs 252. When the MLMs are firstly trained, the output (MLM analysis results 255) includes the models' accuracy. A top-ranked prediction identification (third) operation 246 identifies and selects the top-ranked MLM(s) demonstrating a highest accuracy and performance. The MLMs 252 with the top-ranked accuracy then screens the entire combinatorial library to give top-ranked predictions on the combinations of elements (top-ranked combinations). Those prediction are the output of the MLMs 252, and also serve as input for the following experimental screening. The selected MLM(s) is (are) used to screen the entire combinatorial library 300 to predict the combinations of elements that exhibit the highest desired functions/phenotypes. MLM accuracy and performance can be evaluated by using standard ML methods such as the coefficient of determination denoted R2. The output of operation 246 is a set of top-ranked combinations 256 of elements predicted by the selected MLM(s).

The set of top-ranked combinations 256 are usually trapped in suboptimal regions of the combinatorial space 305. The multi MLM analysis carried out in operation 244 is based on a limited experimental data set (the initial data set 302 as processed through the experimental screening operation 242) and can be biased by the location of the data within the combinatorial space 305. As noted above, the optimal combination of elements as represented by the optimal solution 322 for the desired function/output may be located outside of these suboptimal regions.

To avoid being trapped in these suboptimal regions, and to enhance the breadth of input data distribution, an augmented combinatorial data generator operation 247 is provided that generates random combinations of elements from the combinatorial space 305 and prioritizes under-represented regions of the combinatorial space. One example of a data generator might use a random number generator that gives higher probability for certain pairs/elements. For example, the generator may randomly pick a number from the set {1,2,3}, with specified probabilities of: p(x=1)=0.1, p(x=2)=0.7, p(x=3)=0.2. In this example, the value of 2 is prioritized.

This creates the model-driven augmented combinatorial data 258 that is extracted from poorly explored subregions of the combinatorial space, and promotes consideration/analysis of balanced and broadly distributed combinations that are then used as the model-driven augmented input data 258, along with top-ranked combinations 256 from operation 246. The augmented combinatorial data generator operation 247 used to construct input combinations prioritizing under-represented elements often residing in uncharted regions of the combinatorial space 305 ensures the balance of the input data set and the escape from sub-optimal entrapment regions. The input of the augmented combinatorial data generator operation 247 includes all of the real experimentally tested combinations that have been tested in experiments to this point, since those constitute the ground truth. These tested combinations are evaluated to locate the under-represented region(s), although this could take either the form of elements or combinations of elements. Then the algorithm randomly generates combinations in order to enrich those under-represented regions.

In the data selection operation 248 for experimental screening the top ranked combinations 256 and the model-driven augmented combinatorial data 258 and provided as inputs, and an augmented data set 259 is produced. The augmented data set 259 is then used in the next iteration by the experimental screening operation 242 in place of the initial data set 302 that was produced by the initial phase 210. This may involve defining a similarity factor that guides the identification of new samples to be tested experimentally in the next iteration. Once the selected top-ranked combinations 256 and the model-driven augmented data 258 are identified, a normalized similarity factor measured from the top-ranked combinations 256 is calculated and used to determine the amount of model-driven augmented data 258 to be added to the top-ranked combinations 256. The normalized similarity factor calculation may depend on what the data structure looks like. Generally speaking, the calculation may be performed by: 1) vectorizing the input data and calculating the cosine similarity (cosine angle) among the vectors; or 2) turning the data into data points in the multi-dimension spaces, and calculating the Euclidean distances.

This normalized similarity factor measures how similar the top-ranked combinations are to each other. For example: if combination 1 has {A,B,C,D}, combination 2 has {A,B,C,E}, and combination 3 has {A,E,G,H}, then combinations 1 and 2 are more similar to each other, having only one different member, whereas combination 3 differs by two and three dissimilar members. For instance, if the similarity factor is large, then more samples from the augmented combinatorial data 258 are needed. Put differently, if the predictions are too similar, this indicates that the model is converging to a sub-optimal area and thus we more augmented data is needed to help the model to break out of this convergence (i.e., akin to “thinking outside of the box”). An example of a normalized similarity factors is, for example, the normalized Euclidean distance calculated for the top-ranked combinations projected as data points in the multi-dimensional space 256.

Once the final top-ranked combinations and the model-driven augmented data are identified in operation 248, they are synthesized and tested experimentally in the experimental screening operation 242 in place of the initial data set 302. The results of these experiments are then used to train the ML models, as described above, thus starting a new cycle of the iteration phase 240 of the process 200.

To exit the process, some exit criterion is met, such as, e.g., 1) a number of iterations or exceeding some predefined threshold of time, resource limits/cost, and scope; and/or 2) measuring that the iteration converges into the optimal combination, and thus no further improvement can be made.

For exiting, the process 200 breaks out of the iteration phase 240 and into the solution phase 270, where the optimal combination identification operation 272 provides an optimal solution 322 to the original desired output.

The process 200 may fully exploit the interactions between ML predictive models and the experiments. In particular, the former identifies efficiently the subregions of the combinatorial space 305 where experiments should be performed and the latter provides data used to improve the model accuracy. The process 200 thus allows a faster identification of optimal combinations for the optimal solution 322 with only a small amount of input data via the initial data set 302.

### Use Case

The following example use case illustrates an embodiment of the invention in the field of peroxisome-related proteins. While many peroxisome-related proteins are at least partially functionally annotated, rational engineering approaches have limitations since general knowledge of peroxisome biology is incomplete, especially towards factors controlling peroxisome capacity for protein. Testing all the expression of different combinations of known peroxisome-related genes to increase peroxisome capacity would create an immense combinatorial space, too large to directly test. Accordingly, with rational design, one is limited to exploring subregions of this immense combinatorial space, risking that the global optima lies outside this subregion.

To address this, various embodiments disclosed herein employ the iterative ML approaches discussed above in an effective pipeline, leveraging existing methods for engineering protein sequence combinatorial spaces. This pipeline is illustratively used to computationally explore all possible combinations of 25 peroxisome-related genes to predict capacity for a heterologous cargo protein.

In this example use case, the iterative ML pipeline started with a small initial dataset and utilized six different ML models. It then ranked them to identify which of the six ML models performed best. In the first step of the pipeline, ˜100 rationally-designed strains overexpressing various combinations of the 25 peroxisome-related genes suspected to impact peroxisome capacity were experimentally tested. These rationally-designed strains were engineered to optimize one or more properties. In general, to train an algorithm, the training data preferably maximizes variability to provide examples of both good and bad designs that the algorithm can learn from.

Given the inherent challenge of determining a priori which models are most accurate in describing the specific data landscape, six different ML methods were utilized in this use case. This approach leveraged different architectures and learning approaches characteristic of the structures of each of the six models and provided complementary strengths and robustness in predictions. Among these six ML models, two of them consistently showed the highest accuracy based on the R2 of the testing data in each iteration: the first model is based on a combination of Convolutional Neural Networks (CNN) and Long Short-Term Memory (LSTM) networks, herein called CNN+LSTM, and the second model is based on Gradient Boosting Regression (GBR). For CNN+LSTM, this architecture was determined to be best suited for detecting spatial correlations within the input data, as well as capturing the sequential information. GBR is a powerful ML algorithm known for its capability of handling non-linear, complex relationships in the data by iteratively employing new learners to reduce the prediction error.

The top two performing models (CNN+LSTM and GBR) individually screened the entire combinatorial library to predict combinations of gene overexpression that yielded the largest changes to peroxisome capacity. The output predictions could be a combination of any number of genes from 1 to 25, indicating the expansive range of potential outputs. If only the top-ranked predictions are followed during the search process, some areas of the combinatorial space are underexplored in the experimental screening. This could potentially lead to the combinatorial search becoming trapped in local peaks within the data landscape.

In order to prevent this issue, code was implemented that leverages the co-occurrence matrix of genes and generates combinations accordingly to add to the training set. This code prioritized gene combinations with low co-occurrence, thereby promoting a more balanced and broadly distributed combination input space. These computer model designed combinations for poorly explored regions along with the top-ranked predictions were synthesized and empirically tested. In this way, a more diverse and extended range of combinations could be explored while avoiding becoming trapped in local and subregional peaks. The results of these experiments were then used to again train our ML models, thus starting a new cycle of the iterative ML pipeline.

Through this iterative ML pipeline, the model accuracy was further refined by incorporating newly obtained data and identified novel combinations with higher peroxisome capacity. This eventually allowed discovery of a combination that achieved a higher peroxisome functional capacity than the rational design strain. In the use case, the R2 values of the CNN+LSTM and GBR models were 0.82 and 0.84 for training and 0.69 and 0.62 for testing, respectively. Both models converged on predictions that always included expression of a core set of Pex2p, Pex5p, Pex6p, Pex8p, Pex10p, Pex15p, and Pex22p and sometimes included a supplemental combination of Pex1p, Pex3p, Pex11p, Pex12p, Pex14p, and Pex17p. Different iterations of these gene sets were tested by expressing the core set of genes and adding one gene from the supplemental list.

The highest capacity strain included overexpression of various peroxisome biogenesis factors. This strain had a higher capacity than the rational overexpression of peroxisome complexes and when naturally induced. This combination of genes predicted by the ML methods would not have been chosen based on function of the proteins, as these proteins consist only of subsets of different peroxisome complexes. This enhanced peroxisome capacity (EPC) strain led to an increase of functional capacity of 137%, 39%, and 21% compared to the starting strain, mimicking oleate induction, and our rational engineering strain, respectively. The prediction was able to decrease the number of proteins expressed while increasing the capacity past rational engineering approaches.

This machine learning predicted strain, herein referred to as the EPC strain, provides increased capacity and import rate, as measured via a second, orthogonal method. Previously, it was found that the expression of a truncated norcoclaurine synthase (INCS), which catalyzes the first committed step in the synthesis of benzylisoquinoline alkaloids, is toxic in S. cerevisiae and that localizing the protein to the peroxisome alleviates this toxicity. This step is rate-limiting in the pathway, so high expression of the protein is required for high titers. Accordingly, further increasing peroxisome capacity and import rate enables higher titers to be achieved without sacrificing host cell viability.

When tNCS was expressed in the EPC strain, growth was considerably improved compared to the WT and even the rational overexpression strain, consistent with increased compartmentalization of this toxic protein. The improvement in functional capacity over the rationally-engineered strain shows that even 21% increases to functional capacity led to large functional differences of the peroxisomes. Additionally, the EPC strain enabled faster import into the peroxisome, as demonstrated by a protease competition assay. This illustrates the value of using multiple orthogonal methods to assess both capacity and rate of import, and that any increase to functional capacity leads to meaningful biological improvements.

Utilizing the EPC strain enabled a higher titer of geraniol than WT-capacity peroxisomes. The EPC strain produces higher titers of geraniol when the entire pathway is targeted to the peroxisome than strains with WT peroxisome capacities or strains with the pathway cytosolically compartmentalized. Accordingly, the improved compartmentalization prevented cross-talk and improved flux through the mevalonate pathway while providing access to the native peroxisomal pool of AcCoA. Utilizing the EPC strain led to 80% improvement in titers compared to WT after 24 hours.

By using an embodiment of the machine learning pipeline described herein, it was possible to identify a minimal set of proteins to overexpress that led to increased peroxisome functional capacity and changes to peroxisome biology. This iterative machine learning pipeline was able to explore the combinatorial space of overexpressing all of the different peroxisome-related proteins thought to have an impact on capacity. This iterative pipeline utilized multiple machine learning architectures and ultimately produced the highest capacity strain tested. The best-performing prediction included the overexpression of Pex2p, Pex5p, Pex6p, Pex8p, Pex10p, Pex15p, Pex17p, and Pex22p. This combination of gene overexpressions would not have been predicted by rational engineering.

### TECHNICAL APPLICATION

The one or more embodiments disclosed herein accordingly provide an improvement to technology, namely, to improving the discovery of an optimum solution for a desired function/output in a large combinatorial space of elements.

What is claimed is:

1. A computer-implemented method of exploring a combinatorial space using one or more processors, the method comprising:
identifying a combinatorial library and a desired output;
identifying, from the combinatorial library, an initial dataset to be tested experimentally to create the combinatorial space from elements within the combinatorial library;
iteratively performing:
experimentally screening a set of diverse machine learning (ML) models (MLMs) using the initial data set or an augmented data set to produce experimental screening results that comprise experimental outputs;
training the MLMs using the experimental screening results;
selecting, from the MLMs, a selected MLM set (SMS) comprising at least one MLM having a highest accuracy and performance;
obtaining a set of top-ranked combinations by screening the combinatorial library using the SMS;
calculating a normalized similarity factor measured from the top-ranked combinations;
identifying, using the normalized similarity factor, an amount of model-driven augmented data to be added to the top-ranked combinations;
obtaining a set of model-driven augmented data; and
selecting the model-driven augmented data set from the top-ranked combinations and augmented combinatorial data;
upon meeting an exit criterion:
determining an optimal combination identification that produces the desired output.

2. The computer-implemented method of claim 1, wherein the MLMs comprise scikit-learn library models and customized deep learning models.

3. The computer-implemented method of claim 1, wherein the initial data set is selected from the group consisting of genes, proteins, and chemical compounds.

4. The computer-implemented method of claim 1, wherein combinatorial elements of the combinatorial space are related to an experimentally measurable magnitude.

5. The computer-implemented method of claim 1, wherein the experimental outputs are Boolean values.

6. The computer-implemented method of claim 1, wherein the selecting of the SMS uses an R2 coefficient of determination.

7. The computer-implemented method of claim 1, wherein the obtaining of the set of model-driven augmented data comprises generating random combinations of elements from the combinatorial space that prioritize under-represented areas of the combinatorial space.

8. The computer-implemented method of claim 7, wherein the generating of the random combinations of elements comprises using a random number generator that gives a higher probability for certain elements or combinations of elements.

9. The computer-implemented method of claim 1, wherein the normalized similarity factor is determined by vectorizing input data and calculating a cosine similarity among the vectors.

10. The computer-implemented method of claim 1, wherein the normalized similarity factor is a normalized Euclidean distance calculated for the set of top-ranked combinations.

11. The computer-implemented method of claim 1, wherein the exit criterion include a predefined threshold selected from the group consisting of a number of iterations, a time limit, a scope limit, and a resource limit.

12. The computer-implemented method of claim 1, wherein the exit criterion is determined by measuring a convergence to an optimal combination in which no further improvement can be made.

13. A system for exploring a combinatorial space, comprising:
a memory; and
one or more processors that are configured to:
identify a combinatorial library and a desired output;
identify, from the combinatorial library, an initial dataset to be tested experimentally to create the combinatorial space from elements within the combinatorial library;
iteratively perform:
experimentally screen a set of diverse machine learning (ML) models (MLMs) using the initial data set or an augmented data set to produce experimental screening results that comprise experimental outputs;
train the MLMs using the experimental screening results;
select, from the MLMs, a selected MLM set (SMS) comprising at least one MLM having a highest accuracy and performance;
obtain a set of top-ranked combinations by screening the combinatorial library using the SMS;
calculate a normalized similarity factor measured from the top-ranked combinations;
identify, using the normalized similarity factor, an amount of model-driven augmented data to be added to the top-ranked combinations;
obtain a set of model-driven augmented data; and
select the augmented data set from the top-ranked combinations and the augmented data;
upon meeting an exit criterion:
determine an optimal combination identification that produces the desired output.

14. The system of claim 13, wherein the initial data set is selected from the group consisting of genes, proteins, and chemical compounds.

15. The system of claim 13, wherein the combinatorial elements of the combinatorial space are related to an experimentally measurable magnitude.

16. The system of claim 13, wherein, for the obtainment of the set of model-driven augmented data, the processor is configured to generate random combinations of elements from the combinatorial space that prioritize under-represented areas of the combinatorial space.

17. The system of claim 16, wherein the generation of the random combinations of elements comprises using a random number generator that gives a higher probability for certain pairs or elements.

18. The system of claim 13, wherein the normalized similarity factor is determined by having the processor vectorize input data and calculate a cosine similarity among the vectors or determining a normalized Euclidean distance calculated for the set of top-ranked combinations.

19. The system of claim 13, wherein the exit criterion include at least one of:
a predefined threshold selected from the group consisting of a number of iterations, a time limit, a scope limit and a resource limit; and
a determination by the processor that measures a convergence to an optimal combination in which no further improvement can be made.

20. A computer program product for a system for exploring a combinatorial space apparatus, the computer program product comprising one or more computer readable storage media, and program instructions collectively stored on the one or more computer readable storage media, the program instructions comprising program instructions to:
identify a combinatorial library and a desired output;
identify, from the combinatorial library, an initial dataset to be tested experimentally to create the combinatorial space from elements within the combinatorial library;
iteratively perform:
experimentally screen a set of diverse machine learning (ML) models (MLMs) using the initial data set or an augmented data set to produce experimental screening results that comprise experimental outputs;
train the MLMs using the experimental screening results;
select, from the MLMs, a selected MLM set (SMS) comprising at least one MLM having a highest accuracy and performance;
obtain a set of top-ranked combinations by screening the combinatorial library using the SMS;
calculate a normalized similarity factor measured from the top-ranked combinations;
identify, using the normalized similarity factor, an amount of model-driven augmented data to be added to the top-ranked combinations;
obtain a set of model-driven augmented data; and
select the augmented data set from the top-ranked combinations and the augmented data;
upon meeting an exit criterion:
determine an optimal combination identification that produces the desired output.
